(19) United States
(12) Patent Application Publication
(10) Pub. No.: US 2008/0047030 A1
(43) Pub. Date: Feb. 21, 2008

(54) Plant Dwarfing Gene

(75) Inventors: Minami Matsui, Kanagawa (JP); Miki Nakazawa, Kanagawa (JP); Takanari Ichikawa, Kanagawa (JP); Shu Muto, Tokyo (JP)

Correspondence Address: BIRCH STEWART KOLASCH & BIRCH, PO BOX 747, FALLS CHURCH, VA, 22040-0747, US

(73) Assignee: RIKEN, Wako-shi (JP)

(21) Appl. No.: 10/571,173

(22) PCT Filed: Sep. 10, 2004
(86) PCT No.: PCT/JP04/13594

§ 371 Date: Jan. 16, 2007

(30) Foreign Application Priority Data
Sep. 12, 2003 (JP) 2003-321497

(51) Int. Cl. A01H 5/00; C07H 21/04; C07K 14/00; C12N 15/00; C12N 15/82

(52) U.S. Cl. 800/278; 435/320.1; 530/350; 536/23.6; 800/298; 800/306; 800/317

(57) ABSTRACT

An object of the present invention is to explore, isolate, and identify a dwarfing gene by activation tagging. The present invention provides a gene encoding a protein of the following (a) or (b): (a) a protein consisting of the amino acid sequence represented by any of SEQ ID NOs: 2, 4, 6, and 8; or (b) a protein consisting of an amino acid sequence derived from the amino acid sequence represented by any of SEQ ID NOs: 2, 4, 6, and 8 with the deletion, substitution, or addition of one or several amino acids and having the activity of dwarfing a plant body.

### TECHNICAL FIELD

The present invention relates to a gene having the function of dwarfing a plant, a transformed plant with the gene introduced therein, and so on.

### BACKGROUND ART

Mutants that link genes with phenotypes can be used for identifying gene function. There exist two types of mutants, loss-of-function mutants and gain-of-function mutants.

Loss-of-function mutations have been used for identifying gene function in many organisms. Many mutants were also characterized in Arabidopsis thaliana in the past few years, and most of them result from functionally recessive or loss-of-function mutations. The genomic sequence of Arabidopsis thaliana was determined, and it has been clear that most of its genes belong to families or have sequences closely related on the genome.

Activation tagging is a technique to analyze unknown gene function, which has been developed for Arabidopsis thaliana, and makes it possible to obtain gain-of-function mutants. T-DNA tagging vectors used in activation tagging have enhancers derived from cauliflower mosaic virus (CaMV) 35S promoter and was originally developed by Walden et al (Hayashi, H. et al., Science, 258, 1350-1353, 1992). T-DNA contains the CaMV 35S enhancers tandem arranged at a site proximal to the right border. The Agrobacterium-mediated introduction of this T-DNA into the plant genome activates and overexpresses genes near the insertion site by the action of the enhancers, with the result that a change occurs in the phenotype of the plant body. For example, a gene involved in hormone signaling (Kakimoto, T. et al., Science, 274, 982-985, 1996) and a gene involved in early flowering phenotypes (Kardailsky, I. et al., Science, 286, 1962-1965, 1999) have previously been reported to be isolated from Arabidopsis thaliana by utilizing this activation tagging.

Activation tagging has such advantages that: (i) all activation-tagged mutants are dominant and therefore permit the screening of phenotypes in the T₁ generation; (ii) mutations in redundantly acting genes can be expected to produce phenotypes; and (iii) there is a high possibility of linking activated genes with dominant mutant phenotypes.

Following the development of transformants of Arabidopsis thaliana by use of vacuum infiltration or flower dipping technique, this T-DNA was introduced into plant bodies to create mutant lines or activation-tagged lines. The activation of genes by the CaMV 35S enhancers brings about dominant phenotypes. Several dominant mutants have previously been isolated from activation-tagged lines, and some of them have been shown to be caused by mutations in genes belonging to families. The present inventors have already isolated a dominant mutant dfl1-D involved in auxin response and photomorphogenesis as well as the corresponding gene to this mutation that encodes a soybean GH3 homologue, from the screening of activation-tagged lines of Arabidopsis thaliana (Nakazawa; M. et al., Plant J. 25, 213-221, 2001; and JP Patent Publication (Kokai) No. 2002-10786).

An object of the present invention is to explore, isolate, and identify a dwarfing gene by activation tagging.

### DISCLOSURE OF THE INVENTION

The present inventors screened morphological mutants from the T₁ generations of approximately 50,000 activation-tagged lines of Arabidopsis thaliana and successively isolated genes associated with plant dwarfing, thereby completing the present invention.

Namely, the present invention encompasses the following inventions.

(1) a gene encoding a protein of the following (a) or (b):

(a) a protein consisting of the amino acid sequence represented by any of SEQ ID NOs: 2, 4, 6, and 8; or

(b) a protein consisting of an amino acid sequence derived from the amino acid sequence represented by any of SEQ ID NOs: 2, 4, 6, and 8 with the deletion, substitution, or addition of one or several amino acids and having the activity of dwarfing a plant body.

(2) a gene comprising DNA of the following (c) or (d):

(c) DNA consisting of the nucleotide sequence represented by any of SEQ ID NOs: 1, 3, 5, and 7; or

(d) DNA hybridizing under stringent conditions to DNA consisting of a nucleotide sequence complementary to DNA consisting of the nucleotide sequence represented by any of SEQ ID NOs: 1, 3, 5, and 7 and encoding a protein having the activity of dwarfing a plant body.

(3) a protein of the following (a) or (b):

(a) a protein consisting of the amino acid sequence represented by any of SEQ ID NOs: 2, 4, 6, and 8; or

(b) a protein consisting of an amino acid sequence derived from the amino acid sequence represented by any of SEQ ID NOs: 2, 4, 6, and 8 with the deletion, substitution, or addition of one or several amino acids and having the activity of dwarfing a plant body.

(4) a recombinant vector comprising a gene according to (1) or (2).

(5) a transformed plant with a gene according to (1) or (2) or a recombinant vector according to (4) introduced therein.

(6) the transformed plant according to (5), wherein the plant is a plant body, plant organ, plant tissue, or cultured plant cell.

(7) the transformed plant according to (5) or (6), wherein the plant belongs to any family selected from the group consisting of the families Brassicaceae, Solanaceae, Poaceae, and Leguminosae.

(8) a method for dwarfing a plant body by inducing the overexpression of a gene according to (1) or (2) in the plant body.

### BRIEF DESCRIPTION OF THE DRAWINGS

FIG. 1 shows a map of a T-DNA insertion site in the genome of a tagged line Z029218.

FIG. 2 shows a map of a T-DNA insertion site in the genome of a tagged line Z043547.

FIG. 3 shows a map of a T-DNA insertion site in the genome of a tagged line Z093001.

FIG. 4 shows a map of a T-DNA insertion site in the genome of a tagged line Z029732/Z068035.

FIG. 5 shows a photograph of an At4g31910 transformant (6-week old).

FIG. 6 is a photograph showing the comparison (6-week old) between an At4g31910 transformant (right) and a wild strain (left).

FIG. 7 shows a photograph of an At1g04910 transformant (6-week old).

FIG. 8 shows a photograph of an A1g04910 transformant (6-week old).

FIG. 9 shows a photograph of an At4g35700 transformant (6-week old).

FIG. 10 shows a photograph of an At4g35700 transformant (6-week old).

FIG. 11 shows a photograph of an At1g49770 transformant (6-week old).

FIG. 12 shows a photograph of an At1g49770 transformant (6-week old).

FIG. 13 shows a photograph of separately dried wild-type and At1g49770 transformant seeds (OX-1 to -8 denote the designations of At1g49770 transformant lines; and the left and the right in each panel show the wild-type seeds (controls) and the At1g49770 transformant seeds, respectively).

FIG. 14 shows a result of tannin detection by the DMACA staining of At1g49770 transformant seeds (OX-1 to 8 denote the At1g49770 transformant seeds; and Col denotes wild-type seeds (controls)).

Hereinafter, the present invention will be described in detail. The present application claims the priority of Japanese Patent Application No. 2003-321497 filed on Sep. 12, 2003 and encompasses contents described in the specification and/or drawings of the patent application.

b 1. Isolation of Gene of the Present Invention
(1) Activation Tagging Technique

A gene of the present invention can be obtained by producing transcriptionally activated mutants of plant genes by activation tagging technique, and cloning the causative genes.

Specifically, it can be achieved by the following procedures:
(i) an activation-tagging T-DNA vector is randomly inserted through Agrobacterium into the genome of Arabidopsis thaliana to produce activation-tagged lines;
(ii) T₁ plants are grown from seeds harvested from the tagged lines, and their phenotypic traits are recorded on the basis of predetermined inspection items for phenotypic traits, simultaneously with digital imaging thereof;
(iii) DNA fragments containing the T-DNA are collected by plasmid rescue from the genomes of the mutant strains whose phenotypic traits clearly differ in the T₁ generation from those of wild types, and are then sequenced;
(iv) the DNA fragments are introduced into wild-type Arabidopsis thaliana to examine whether the mutant phenotypic trait can be reproduced; and
(v) the corresponding cDNA is cloned.

In the present specification, the “T₁ generation” means a plant generation obtained from seeds of plants of the “T₀ generation”, a transformed plant generation. The “T₁ generation” is the first population from the transformed plants and can be selected using selective agents (e.g., antibiotics and herbicides) compatible with the resistant genes of the transgenic plants. Alternatively, the “T₂ generation” means a plant generation obtained by the self-pollination of flowers of the “T₁ generation”0 plants preselected as transgenic plants.

pPCVICEn4HPT developed by Walden et al (Hayashi, H. et al, Science, 258, 1350-1353, 1992) can be used as the activation-tagging T-DNA vector. This vector is a binary vector that tandem possesses 4 enhancers (-90 to -440) in CaMV 35S promoter near the RB. Arabidopsis thaliana is transformed with Agrobacterium GV3101 (pMP90RK) bearing this pPCVICEn4HPT. The transformation can be performed by flower dipping technique that involves immersing and coculturing the above ground part of Arabidopsis thaliana in a suspension of Agrobacterium.

Examples of phenotypic traits observed in the T₁ plants include plant body height, rosette color, the number of leaves before bolting, leaf width, petiole length, flower shape, the number of floral organs, flowering time, and the fertility and blanching of shoots.

### (2) Plasmid Rescue Technique and Functional Assay of Resulting DNA Fragments

If interesting mutants are obtained, genes that cause the mutations by transcriptional activation are cloned. Plasmid rescue technique is preferred as a method for the cloning. Specifically, DNAs of the mutants are purified and treated with a variety of restriction enzymes. Band size is confirmed by southern blot to find restriction enzymes that offer approximately 10- to 20-kb fragments containing the inserted T-DNA. Next, the DNAs are treated with those restriction enzymes and subsequently with phenol/chloroform. The resulting DNAs are precipitated with ethanol and then self-ligated with ligase. These DNAs are introduced by electroporation into competent cells (Escherichia coli DH10B). After the selection of resistant strains on a medium containing ampicillin, plasmids are selected by a routine method. A genomic DNA boundary sequence adjacent to the inserted T-DNA contained in the obtained plasmid is determined, and the position of T-DNA insertion on the genome is determined. Based on the position, genes having translation initiation sites within 6 kb from the enhancer sequences are searched using Arabidopsis thaliana genome database (http://www.mips.biochem.mpg.de). These genes are used as candidate genes to design primers specific to the genes or recombinant vectors introduced into the plants. cDNAs are amplified from an Arabidopsis thaliana cDNA library, and the resulting amplified fragments are cloned. These cDNA fragments are introduced through Agrobacterium into plants to examine whether the mutant phenotype is reproduced.

Although cDNA sequencing can be performed by an approach known in the art such as Maxam-Gilbert chemical modification method or dideoxynucleotide chain termination method using M13 phage, cDNA is typically sequenced using an automatic sequencer (e.g., ABI 373 sequencer and 310 DNA sequencer; both manufactured by Applied Biosystems). The obtained nucleotide sequences are analyzed with DNA analysis software such as DNASIS (Hitachi Software Engineering), and coding regions for encoded proteins can be found in the resulting DNA strands.

The gene of the present invention isolated and identified by the approach described above is At4g31910 (Z029218), At1g04910 (Z043547), At4g35700 (Z093001), and At1g49770 (Z029732, Z068035) (tagged-line designations are given in the parentheses). The nucleotide sequences of At4g31910, At1g04910, At4g35700, and At1g49770 are shown in SEQ ID NOs: 1, 3, 5, and 7, respectively. Alternatively, amino acid sequences encoded by At4g31910, At1g04910, At4g35700, and At1g49770 are shown in SEQ ID NOs: 2, 4, 6, and 8, respectively.

As long as a protein consisting of each amino acid sequence represented by any of SEQ ID NOs: 2, 4, 6, and 8 can dwarf a plant body, the amino acid sequence may have the mutations such as deletion, substitution, and addition of plural amino acids, preferably one or several amino acids.

For example, 1 to 10 amino acids, preferably 1 to 5 amino acids, may be deleted in the amino acid sequence represented by any of SEQ ID NOs: 2, 4, 6, and 8, may be added to the amino acid sequence represented by any of SEQ ID NOs: 2, 4, 6, and 8, or may be substituted in the amino acid sequence represented by any of SEQ ID NOs: 2, 4, 6, and 8 by other amino acids.

The scope of the present invention also encompasses a gene that encodes a protein having 70% or more homology to the amino acid sequence represented by any of SEQ ID NOs: 2, 4, 6, and 8 and having the activity of dwarfing a plant body. The 70% or more homology refers to preferably 80% or more homology, more preferably 90% or more homology, most preferably 95% or more homology.

The deletion, addition, and substitution of the amino acids can be performed by modifying the gene encoding the protein by an approach known in the art. The mutations can be introduced into the gene by an approach known in the art such as Kunkel or Gapped duplex method or an equivalent method thereof. For example, a mutation-introducing kit utilizing site-specific mutagenesis (e.g., Mutant-K (manufactured by TAKARA) and Mutant-G (manufactured by TAKARA)) is used, or alternatively LA PCR in vitro Mutagenesis Series Kit available from TAKARA is used, to introduce the mutations.

In this context, the “activity of dwarfing a plant body” in the present invention means the activity of decreasing the height of a plant body (e.g., Arabidopsis thaliana) with the expression of the gene of the present invention to ⅔ to 1/10, preferably ⅓ to 1/10 those of wild types. The phrase “having the activity of dwarfing a plant body” means that the activity described above is substantially equal to the activity of the protein having the amino acid sequence represented by any of SEQ ID NOs: 2, 4, 6, and 8.

The gene of the present invention also encompasses DNA hybridizing under stringent conditions to DNA consisting of a nucleotide sequence complementary to DNA consisting of the nucleotide sequence represented by any of SEQ ID NOs: 1, 3, 5, and 7 and encoding a protein having the activity of dwarfing a plant body. In this context, the stringent conditions refer to conditions that provide for the formation of so-called specific hybrids but no nonspecific-hybrid. Examples of the stringent conditions include conditions under which the complementary strand of a nucleic acid having high homology, that is, DNA consisting of a nucleotide sequence having 90% or more homology, preferably 95% or more homology, to the nucleotide sequence represented by any of SEQ ID NOs: 1, 3, 5, and 7, is hybridized, while the complementary strand of a nucleic acid having lower homology is not hybridized. To be more specific, the stringent conditions refer to conditions at a sodium concentration of 15 to 300 mM, preferably 15 to 75 mM, and at a temperature of 50 to 60° C., preferably 55 to 60° C.

Once the nucleotide sequence of the gene of the present invention is definitely determined, the gene of the present invention can be obtained by chemical synthesis, by PCR using cloned cDNA as a template, or by the hybridization of a DNA fragment having the nucleotide sequence as a probe. In addition, modified DNA that encodes the gene can be synthesized by site-specific induction or the like.

### 2. Production of Recombinant Vector and Transformed Plant

### (1) Production of Recombinant Vector

A recombinant vector of the present invention can be produced by inserting the gene of the present invention into an appropriate vector. pBI-, pUC-, and pTRA-type vectors are preferably used as vectors for introducing and expressing the gene of the present invention into a plant cell. The pBI- and pTRA-type vectors allow the introduction of genes of interest into plants through Agrobacterium. pBI-type binary or intermediate vectors are preferably used, and examples thereof include pBI121, pBI101, pBI101.2, and pBI101.3. The pUC-type vectors allow the direct introduction of genes into plants, and examples thereof include pUC18, pUC19, and pUC9. Alternatively, plant virus vectors such as cauliflower mosaic virus (CaMV), bean golden mosaic virus (BGMV), tobacco mosaic virus (TMV) can also be employed.

For inserting the gene of the present invention into the vector, for example, a method is adopted in which purified DNA is initially cleaved with an appropriate restriction enzyme and then inserted into the restriction enzyme site or multicloning site of appropriate vector DNA, which is in turn ligated with the vector.

The gene of the present invention should be incorporated into the vector so that its gene function is exhibited. Therefore, the vector can be ligated, if desired, with an enhancer, splicing signal, poly(A)-addition signal, selective marker, 5′-UTR sequence, and so on, in addition to a promoter and the gene of the present invention. Examples of the selective marker include dihydrofolate reductase gene, ampicillin-resistant gene, neomycin-resistant gene, hygromycin-resistant gene, and bialaphos-resistant gene.

The “promoter”0 does not have to be derived from plants, as long as it is DNA that can function in plant cells and induce gene expression in particular plant tissues or in particular plant developmental stages. Concrete examples of the promoter include cauliflower mosaic virus (CaMV) 35S promoter, nopaline synthase gene promoter (Pnos), maize-derived ubiquitin promoter, rice-derived actin promoter, and tobacco-derived PR protein promoter.

A “terminator”0 may be a sequence that can terminate the transcription of genes transcribed by the aforementioned promoter. Concrete examples of the terminator include nopaline synthase gene terminator (Tnos) and cauliflower mosaic virus poly(A) terminator.

The “enhancer” is used for increasing the expression efficiency of genes of interest. For example, an enhancer region containing an upstream sequence in the CaMV 35S promoter is preferable.

### (2) Production of Transformed Plant

A transformed plant of the present invention can be obtained by introducing the gene of the present invention or the recombinant vector containing it into a host so as to allow the expression of the gene of interest. In this contest, the host is not particularly limited as long as it can express the gene of the present invention. However, a plant is preferable as the host. When the host is a plant, the transformed plant (transgenic plant) can be obtained in the way described below.

The plant to be transformed in the present invention means any of an entire plant body, a plant organ (e.g., leaves, petals, stems, roots, and seeds), a plant tissue (e.g., epidermis, phloem, parenchyma, xylem, and vascular bundle), and a cultured plant cell.

Examples of the plant used in transformation include, but not limited to, plants belonging to the families Brassicaceae, Poaceae, Solanaceae, and Leguminosae (see the list described below).
Family Brassicaceae: Arabidopsis thaliana
Family Solanaceae: Nicotiana tabacum
Family Poacea: Zea mays and Oryza sativa
Family Leguminosae: Glycine max

Examples of a method for introducing the gene or recombinant vector of the present invention into the plant include Agrobacterium, PEG-calcium phosphate, electroporation, liposome, particle gun, and microinjection techniques. For example, when the Agrobacterium technique is used, this technique utilizes protoplasts in some cases and tissue sections in other cases. When a protoplast is used, the protoplast is cultured together with the Agrobacterium having a Ti plasmid, or it is fused with a spheroplasted Agrobacterium (the spheroplast method). When a tissue section is used, Agrobacterium is allowed to infect an aseptically cultivated leaf section (a leaf disc) of target plant (the leaf disc method) or a callus (undifferentiated cultured cell).

Confirmation as to whether or not the gene is incorporated into the plant can be performed by PCR, southern hybridization, northern hybridization, or the like. For example, DNA is prepared from the transformed plant and subjected to PCR using designed DNA-specific primers. After PCR, the resulting amplification product is subjected to agarose gel electrophoresis, polyacrylamide gel electrophoresis, or capillary electrophoresis, or the like, and then stained with ethidium bromide, SYBR Green solution, or the like. The amplification product can be detected as a single band to thereby confirm that the transformation is successful. Alternatively, the amplification product can also be detected by performing PCR using primers labeled in advance with fluorochrome or the like. Furthermore, a method may be used in which the amplification product is bound to a solid phase such as microplates and confirmed by, for example, fluorescent or enzyme reaction.

Tumor tissues, shoots, hairy roots, and so on, obtained by transformation may be used directly in cell culture, tissue culture, or organ culture, or can be regenerated into plant bodies using a conventionally known plant tissue culture method by, for example, the administration of plant hormone (e.g., auxin, cytokinin, gibberellin, abscisic acid, ethylene, and brassinolide) with an appropriate concentration.

Although the transformed plants thus obtained are typically “dwarfed”, they respectively have phenotypic traits described below.
At4g31910: small rosettes, round leaves, epinastic leaves, short petiole, short internodal spacings, the second inflorescence having a wide angle relative to the first inflorescence
At1g04910: small leaves, epinastic leaves, abnormal internodal spacings
At4g35700: small rosettes, round leaves, short internodal spacings
At1g49770: decreased apical dominance, short internodal spacings, decreased tannin contents

In the present specification, the term “phenotype” is used synonymously with the term “phenotypic trait”. These terms mean the easily observable or measurable morphological characteristics of plants.

The phenotypic trait of a short height has such advantages that: plants having this phenotypic trait are resistant to wind damage such as typhoons and are less likely to fall down even by increased grains; for example, in the case of rice, the density of seedling planting per unit area can be rendered larger because the number of rows for seedlings to be planted can be increased; and when it is applied to fruit trees (e.g., banana and mango) and palm trees (e.g., date and coconut) that extend to a few meters in height, labors for harvesting fruits are alleviated, and yields per unit resource (e.g., water and fertilizers) are increased.

The phenotypic trait of unconventional leave or flower shape or size has such advantages as enhanced commercial value in cut flowers, foliage plants, and bonsai plants, and attractiveness to purchasers.

For example, At4g31910 is dwarfed and however, has a feature of the absence of decreases in the number of flowers and yields. It has advantages from both standpoints: horticulturally, the enhanced clustering of flowers, and agriculturally, dwarfing and the maintenance of yields.

Moreover, At1g49770 has a feature of decreased tannin contents. However, this should be noted in that there has been no report on means that can negatively control the expression level of tannin. For example, this feature allows the control of flower color, reduction in astringent taste, the inhibition and suppression of iron absorption, and so on.

### 3. Production of Protein of the Present Invention

A protein of the present invention can be obtained by ligating (inserting) the gene of the present invention isolated in the paragraph 1 into a recombinant vector such as plasmid DNA and phage DNA capable of replication in hosts, and then introducing the vector into a host such as Escherichia coli other than plant hosts to give a transformant, which is in turn cultured, followed by collection from the resulting cultured product. In this contest, the “cultured product” means any of a culture supernatant, a cultured cell, a cultured microorganism, or a disrupted product of the cultured cell or microorganism.

Examples of the plasmid DNA include Escherichia coli-derived plasmids (e.g., pBR322, pBR325, pUC118, pUC119, pUC18, pUC19, and pBluescript), Bacillus subtilis-derived plasmids (e.g., pUB110 and pTP5), and yeast-derived plasmids (e.g., Yep13 and YCp50). Examples of the phage DNA include λ phages (e.g., Charon4A, Charon21A, EMBL3, EMBL4, λgt10, λgt11, and λZAP). In addition, animal viruses such as retroviruses or vaccinia viruses as well as insect virus vectors such as baculoviruses can also be employed.

For example, bacteria belonging to the genus Escherichia such as Escherichia coli, bacteria belonging to the genus Bacillus such as Bacillus subtilis, bacteria belonging to the genus Pseudomonas such as Pseudomonas putida, bacteria belonging to the genus Rhizobium such as Rhizobium meliloti, yeasts such as Saccharomyces cerevisiae and Schizosaccharomyces pombe, animal cells such as COS and CHO cells, or insect cells such as Sf9 can be used as the host other than plant hosts.

When a bacterium such as Escherichia coli or a yeast is used as the host, it is preferred that the recombinant vector described above should be capable of autonomous replication in the bacterium and should be composed of a promoter, a ribosome-binding sequence, the gene of the present invention, and a transcription termination sequence. The recombinant vector may also contain a gene that controls the promoter.

Examples of the Escherichia coli include, but not limited to, Escherichia coli DH5α and HB01. Examples of the Bacillus subtilis include, but not limited to, Bacillus subtilis. In this case, the promoter is not particularly limited as long as it allows gene expression in the host such as Escherichia coli. For example, an Escherichia coli- or phage-derived promoter such as trp promoter, lac promoter, P_{L} promoter, and P_{R} promoter can be used. A method for introducing the recombinant vector into the bacterium is not particularly limited as long as it is a method that provides for the introduction of DNA into a bacterium. Examples thereof include a method that uses calcium ions (Cohen, S. N. et al., Proc. Natl. Acad. Sci., USA, 69: 2110 (1972)) as well as electroporation.

When a yeast is used as the host, for example, Saccharomyces cerevisiae or Pichia pastoris is used. In this case, the promoter is not particularly limited as long as it allows gene expression in the yeast. For example, gall promoter, gal10 promoter, heat shock protein promoter, MFα1 promoter, PHO5 promoter, PGK promoter, GAP promoter, ADH promoter, and AOX1 promoter can be used. A method for introducing the recombinant vector into the yeast is not particularly limited as long as it is a method that provides for the introduction of DNA into a yeast. Examples thereof include electroporation, spheroplast, and lithium acetate methods.

When an animal cell is used as the host, for example, a monkey cell COS-7 or Vero, Chinese hamster ovary (CHO) cell, or murine L cell is used. In this case, SRα promoter, SV40 promoter, LTR promoter, CMV promoter, or the like is used as the promoter, or otherwise, human cytomegalovirus early gene promoter or the like may also be employed as the promoter. Examples of a method for introducing the recombinant vector into the animal cell include electroporation, calcium phosphate, and lipofection methods.

When an insect cell is used as the host, Sf9 cell or the like is used. Examples of a method for introducing the recombinant vector into the insect cell include calcium phosphate, lipofection, and electroporation methods.

Procedures for culturing the transformant described above are performed according to a routine method used in host culture.

A medium used for culturing the transformant obtained from a microorganism such as Escherichia coli or a yeast used as the host may be any of natural and synthetic media as long as it contains carbon sources, nitrogen sources, inorganic salts, and so on capable of being assimilated by the microorganism and allows the efficient culture of the transformant. Examples of the carbon sources include: carbohydrates such as glucose, fructose, sucrose, and starch; organic acids such as acetic acid and propionic acid; and alcohols such as ethanol and propanol. Examples of the nitrogen sources include: ammonia; ammonium salts of inorganic or organic acids such as ammonium chloride, ammonium sulfate, ammonium acetate, and ammonium phosphate; other nitrogen-containing compounds; peptone; meat extracts; and corn steep liquor. Examples of inorganic substances include dipotassium hydrogen phosphate, potassium dihydrogen phosphate, magnesium phosphate, magnesium sulfate, sodium chloride, ferrous sulfate, manganese sulfate, copper sulfate, and calcium carbonate. The transformant is typically cultured at 37° C. under aerobic conditions such as shaking culture or aeration agitation culture. pH adjustment is performed using an inorganic or organic acid, an alkali solution, or the like. During the culture, an antibiotic such as ampicillin or tetracycline may optionally be added to the medium.

When a microorganism transformed with an expression vector using an inducible promoter as the promoter is cultured, an inducer may optionally be added to the medium. For example, when a microorganism transformed with an expression vector having a promoter inducible by isopropyl-β-D-thiogalactopyranoside (IPTG) is cultured, IPTG or the like can be added to the medium. Alternatively, when a microorganism transformed with an expression vector using trp promoter inducible by indoleacetic acid (IAA) is cultured, IAA or the like can be added to the medium.

Examples of a medium used for culturing a transformant obtained from an animal cell used as the host include RPMI 1640 medium and DMEM medium generally used, or these media supplemented with fetal bovine serum. The transformant is typically cultured at 37° C. for 1 to 30 days in the presence of 5% CO₂. During the culture, an antibiotic such as kanamycin or penicillin may optionally be added to the medium.

If the protein of the present invention is produced in the microorganism or cell after the culture, the protein of interest is collected by disrupting the microorganism or cell by ultrasonication, repetitive freeze-thaw, homogenizer treatment, or the like. Alternatively, if the protein of the present invention is produced outside the microorganism or cell, the culture solution is directly used or is subjected to centrifugation or the like to remove the microorganism or cell. Then, the protein of the present invention can be isolated and purified from the cultured product by using, alone or in an appropriate combination, general biochemical methods used in protein isolation and purification, for example, ammonium sulfate precipitation, gel chromatography, ion exchange chromatography, and affinity chromatography.

### 4. Method for Dwarfing Plant Body

The present invention also provides a method for dwarfing a plant body by inducing the overexpression of the gene of the present invention in the plant body.

The method for dwarfing a plant according to the present invention is not particularly limited to methods using transformation and also encompasses, for example, a method that comprises administrating a substance or imparting environmental stress, to a plant in a developmental stage to induce the overexpression of the gene of the present invention, thereby dwarfing the plant.

### BEST MODE FOR CARRYING OUT THE INVENTION

Hereinafter, the present invention will be described more fully with reference to Examples. However, the present invention is not intended to be limited to these Examples.

### EXAMPLE 1

### Production and Gene Analysis of Mutants

### (1) Activation-Tagged Lines and Plant Growth Conditions

An Arabidopsis thaliana wild type (Col-0) was grown at 22° C. in long-day conditions (16-hour light and 8-hour dark conditions) under white fluorescent tubes (FL40SW; manufactured by Sanyo).

Plants were transformed by floral dipping technique with Agrobacterium tumefaciens GV3101 strains (pMP90RK) containing the activation-tagging T-DNA vector, pPCVICEn4HPT, provided by Dr. R. Walden (Clough, S. J. and Bent, A. F., Plant J., 16, 735-743, 1998; and Hayashi, H., et al., Science, 258, 1350-1353, 1992). Hygromycin-resistant T₁ seedlings were selected on a basic agar medium for 7 days containing 1 mM KNO₃, 0.8% agar, and 50 mg/l hygromycin and transferred into soil (Nakazawa, M. and Matsui, M., Biotechniques, 34, 28-30, 2003).

During the growth of T₁ plant, 3 strains of visible phenotype mutants were picked up (tagged lines Z029218, Z043547, and Z093001), from which some rosette leaves were harvsted as a material for plasmid rescue.

### (2) Plasmid Rescue and Identification of T-DNA Insertion Site in the Genome of Arabidopsis thaliana

About 200 mg of the rosette leaves were collected into screw-capped plastic tubes. Five ceramic particles (CERAMICS YTZ ball, D: 3 mm; manufactured by Nikkato) and 300 μl of lysis buffer were added to the plant material, which was then homogenized using Shake Master (manufactured by BioMedical Science). Genomic DNAs were extracted using Wizard Magnetic 96 DNA Plant System (manufactured by Promega). A workstation system (Tecan genesis workstation 150) was used for conducting the extraction protocol described in the extraction kit. The genomic DNAs were digested with BamHI in a volume of 100 μl overnight. The digested DNAs were precipitated with ethanol and dissolved in 8 μl of distilled water. The DNAs were self-ligated with T4 ligase (400 units/μl; manufactured by BioLabs) in a volume of 10 μl at room temperature overnight. Escherichia coli DH10B electro-competent cells (20 μl; manufactured by Invitrogen) were transformed by electroporation with 2 μl of the ligation solution and spread on LB medium containing ampicillin. After incubation at 38° C. overnight, 8 colonies per line were selected and subcultured in 5 ml of LB liquid medium containing ampicillin. Plasmid DNAs were prepared from the resulting liquid medium and digested with HindIII to distinguish plasmids that did not contain genomic fragments.

The genomic fragments in the plasmids were sequenced using LB2 primer (5′-TGACCATCATACCCATTGCTGATCC-3′). The obtained sequencing results were analyzed using the BLASTN program against the NCBI non-redundant database to identify the position of T-DNA insertion sites in the genome of Arabidopsis thaliana.

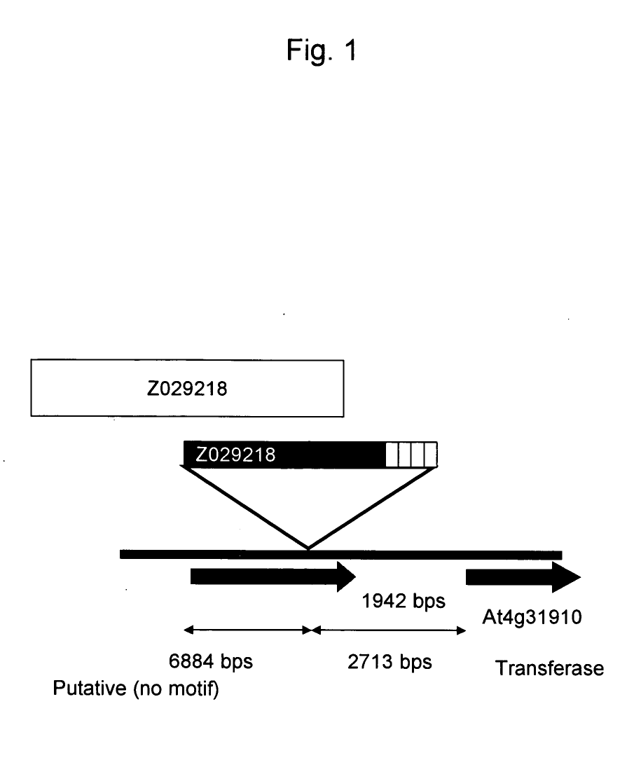

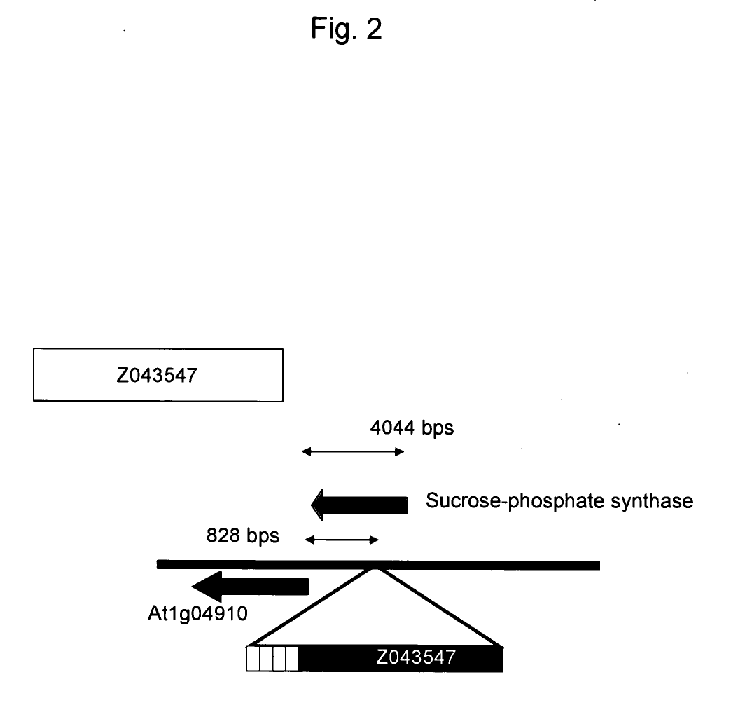

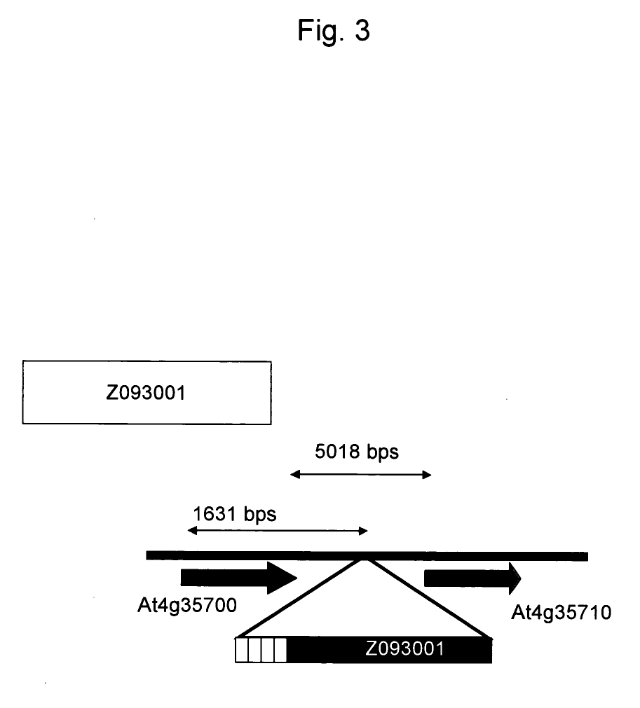

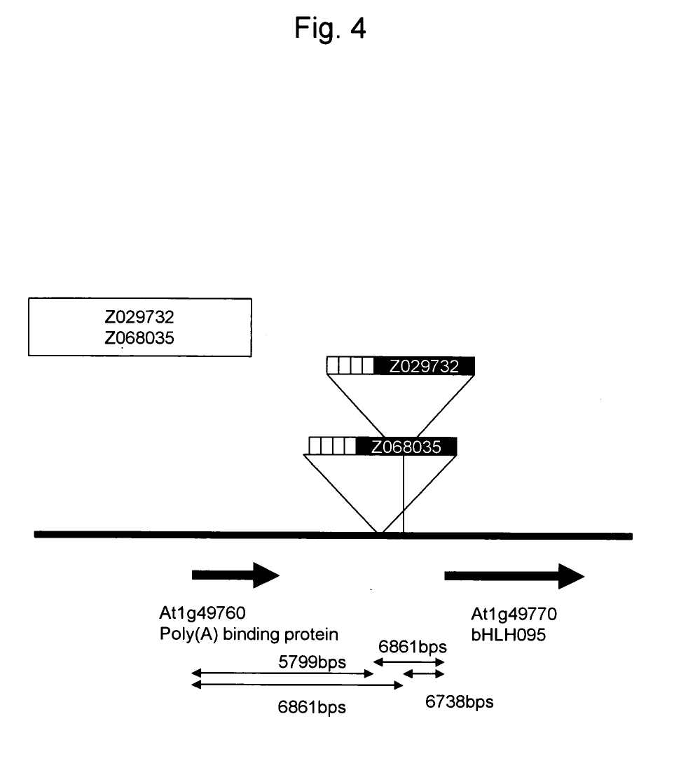

The insertion sites on the genomes of mutants (tagged lines Z029218, Z043547, Z093001, and Z029732/Z068035) are shown in FIGS. 1 to 4, respectively (in FIGS. 1 to 4, thin bars represent genomic DNAs; thick arrows represent candidate genes; and numbers on thin arrows represent the distances between enhancers on T-DNA and the predicted translation initiation sites of the candidate genes).

### (3) Construction of Vectors and Production of Transgenic Plants

The open reading frames of candidate genes (At4g31910, At1g04910, At4g35700, and At1g49770) were amplified by PCR using primers described below from a cDNA library prepared from 5-day-old light-grown seedlings (PCR conditions: 30 cycles of 94° C. for 30 sec., 60° C. for 30 sec., and 72° C. for 8 min).

### (For At4g31910 Amplification)

At4g31910-F:

(SEQ ID NO: 9)

ggggacaagt ttgtacaaaa aagcaggctc gatgcccatg
ttaatggcga cac
At4g31910-R:

(SEQ ID NO: 10)

ggggaccact ttgtacaaga aagctgggtt tagcaatcaa
ggaaatgatt tgaaaagcc

### (For Atg04910 Amplification)

At1g04910-F:

At1g04910-F:

(SEQ ID NO: 11)

ggggacaagt ttgtacaaaa aagcaggctc gatggtggcg
aaactgtcta tcg
At1g04910-R:

(SEQ ID NO: 12)

ggggaccact ttgtacaaga aagctgggtt taccgttcta
atccagtgac cg

### (For At4g35700 Amplification)

At4g35700-F:

At4g35700-F:

(SEQ ID NO: 13)

ggggacaagt ttgtacaaaa aagcaggctc gatgagtaat
cccgagaagt ctaaaattg
At4g35700-R:

(SEQ ID NO: 14)

ggggaccact ttgtacaaga aagctgggtt cactcgactt
tatcatcgtt ctctt

### (For At1g49770 Amplification)

At1g49770-F:

At1g49770-F:

(SEQ ID NO: 15)

ggggacaagtttgtacaaaaaagcaggctatgactaatgctcaagagttg
ggg
At1g49770:R:

(SEQ ID NO: 16)

ggggaccactttgtacaagaaagctgggtttaaagtgaaaagtccaagct
aatc

The amplified fragments were cloned into pDONR-207 vector by BP reaction (recombinant reaction between attB and attB) according to the manual of Gateway PCR Cloning System (manufactured by Invitrogen). After sequence confirmation, the inserts were transferred to pBIDAVL-GWR1 using LR Clonase Enzyme Mix (manufactured by Invitrogen) according to the manufacture's instructions. The “reading frame A cassette” of Gateway Vector Conversion System was replaced with the GUS coding region of pBI121, thereby producing the final vector pBIDAVL-GWR1. The resulting pBIDAVL-GWR1 cDNA was introduced into Agrobacterium tumefaciens GV3101 (pMP90). An Arabidopsis thaliana wild type (Col-0) was transformed with this vector by floral dipping technique. Seeds harvested therefrom were placed on sucrose-free GM plates containing 50 mg/l kanamycin and 100 mg/l cefotaxime. Drug resistant strains were transferred to soil and grown in a greenhouse under the light conditions described above.

FIG. 5 shows a photograph of an At4g31910 transformant (6-week old). The transformant has a low height, short internodal spacings, and short stems that spread sideways. FIG. 6 is a photograph showing the comparison (6-week old) between an At4g31910 transformant and a wild strain. The At4g31910 transformant is about one-third the height of the wild strain.

FIGS. 7 and 8 respectively show a photograph of an At1g04910 transformant (6-week old). The At1g04910 transformant has a height as low as that of the At4g31910 transformant and has a feature of flowers densely clustered at stem tips in stems grown later. In FIG. 8, the trait is severely exhibited.

FIGS. 9 and 10 respectively show a photograph of an At4g35700 transformant (6-week old). Both of them have a low height and barely extend upward. In FIG. 10, the trait is severely exhibited.

FIGS. 11 and 12 respectively show a photograph of an At1g49770 transformant (6-week old). Both of them have a low height and barely extend upward. In FIG. 12, the trait is severely exhibited.

It is noted that the transformants of the genes described above are T₂ generations.

TABLE 1

| | Height | |
|---|---|---|
| Strain | on 6th week | |
| COL-0 | 33 | cm |
| At4g31910 | 10 to 15 | cm |
| At1g04910 | 3 to 15 | cm |
| At4g35700 | 1 to 5 | cm |
| At1g49770 | 1 to 20 | cm |

Moreover, the dried seeds of the At1g49770 transformants were dipped for 1 week in a solution of 2% (w/v) DMACA (p-dimethylaminocinnamaldehyde)/3 M HCl/50% (w/v) methanol to observe black staining caused by the bond between tannin and the DMACA. The dried seeds and a result of tannin detection are shown in FIGS. 13 and 14, respectively. Wild-type seeds (controls) assumed black color, in which tannin was detected, whereas the At1g49770 transformant seeds were lightly stained, in which tannin contents were reduced.

All publications, patents, and patent applications cited herein are incorporated herein by reference in their entirety.

### INDUSTRIAL APPLICABILITY

The present invention provides a gene having the function of dwarfing a plant body. Because a plant transformed with the gene has a height rendered short, the plant does not fall down by increases in the number of grains and has the effect of being resistant to wind damage such as typhoons.

Thus, the present invention can be utilized in yield enhancement, phenotypic trait. diversification, and so on by plant dwarfing.

1. A gene encoding a protein of the following (a) or (b):
(a) a protein consisting of the amino acid sequence represented by any of SEQ ID NOs: 2, 4, 6, and 8; or
(b) a protein consisting of an amino acid sequence derived from the amino acid sequence represented by any of SEQ ID NOs: 2, 4, 6, and 8 with the deletion, substitution, or addition of one or several amino acids and having the activity of dwarfing a plant body.

2. A gene comprising DNA of the following (c) or (d):
(c) DNA consisting of the nucleotide sequence represented by any of SEQ ID NOs: 1, 3, 5, and 7; or
(d) DNA hybridizing under stringent conditions to DNA consisting of a nucleotide sequence complementary to DNA consisting of the nucleotide sequence represented by any of SEQ ID NOs: 1, 3, 5, and 7 and encoding a protein having the activity of dwarfing a plant body.

3. A protein of the following (a) or (b):
(a) a protein consisting of the amino acid sequence represented by any of SEQ ID NOs: 2, 4, 6, and 8; or
(b) a protein consisting of an amino acid sequence derived from the amino acid sequence represented by any of SEQ ID NOs: 2, 4, 6, and 8 with the deletion, substitution, or addition of one or several amino acids and having the activity of dwarfing a plant body.

4. A recombinant vector comprising a gene according to claim 1 or 2.

5. A transformed plant with a gene according to claim 1 or 2 or a recombinant vector according to claim 4 introduced therein.

6. The transformed plant according to claim 5, wherein the plant is a plant body, plant organ, plant tissue, or cultured plant cell.

7. The transformed plant according to claim 5, wherein the plant belongs to any family selected from the group consisting of the families Brassicaceae, Solanaceae, Poaceae, and Leguminosae.

8. A method for dwarfing a plant body by inducing the overexpression of a gene according to claim 1 or 2 in the plant body.


SEQUENCE LISTING (17 sequences)

SEQ ID NO: 1 - DNA, 1377 bp, arabidopsis
  CDS (1)..(1377)
atgcccatgttaatggcgacacgtatcgatataatccaaaagcttaatgtatatccaaggtttcaaaaccatgacaagaagaaactaatcactctctccaatttggaccgtcagtgtcctttactcatgtactctgtcttcttctacaagaataccacaactcgtgactttgactccgtcttctccaacctgaagctcgggctggaggagactatgtctgtgtggtatcccgcggcagggagactgggtttggacggaggtggctgcaagctcaacatccggtgtaacgatggtggcgcagtcatggtggaggcggtggcgacaggtgtcaagttgtccgagcttggtgatttgactcagtacaatgagttttatgagaatttagtttacaagccttccttggatggtgatttctctgtgatgcctcttgttgttgctcaggtgacaagatttgcatgtggaggttactcaattggaatcggtacaagccactctctatttgatggaatctcagcttacgaattcattcacgcgtgggcctccaactctcacattcacaacaaatccaacagcaagattactaataaaaaggaagatgtggtcatcaaaccggttcatgatcgacgaaatctactggttaaccgggatgctgtccgagaaaccaatgctgcagccatttgtcatctgtaccagttgatcaaacaggcgatgatgacctatcaggagcaaaaccgtaacttagagttaccagactctggttttgtgatcaaaacgttcgagcttaatggcgatgcgatagaaagcatgaagaagaaatcactagaagggttcatgtgctcctcctttgagtttcttgctgctcatttgtggaaggcaagaacaagggctttagggttgaggagagacgccatggtgtgtttacaattcgcagtggacataaggaaaagaacggagacaccgctgccagaagggttttccggcaacgcatacgtgcttgcctcggtggcatccaccgccagagaattacttgaagagctaacactcgagtcaatagtcaacaagatcagagaagccaagaaatcaattgaccaaggttacataaactcttacatggaagcactcggaggtagtaatgacggaaatctccctcctctcaaagagctaaccctaatctccgactggacaaaaatgccatttcacaatgttggctttggcaacggcggcgagccagcggattacatggccccactgtgtccaccggtgccacaagttgcttatttcatgaagaaccctaaagatgccaaaggtgttcttgtgaggattggcttggacccacgagatgttaatggcttttcaaatcatttccttgattgctaa

SEQ ID NO: 2 - protein, 458 aa, arabidopsis
MPMLMATRIDIIQKLNVYPRFQNHDKKKLITLSNLDRQCPLLMYSVFFYKNTTTRDFDSVFSNLKLGLEETMSVWYPAAGRLGLDGGGCKLNIRCNDGGAVMVEAVATGVKLSELGDLTQYNEFYENLVYKPSLDGDFSVMPLVVAQVTRFACGGYSIGIGTSHSLFDGISAYEFIHAWASNSHIHNKSNSKITNKKEDVVIKPVHDRRNLLVNRDAVRETNAAAICHLYQLIKQAMMTYQEQNRNLELPDSGFVIKTFELNGDAIESMKKKSLEGFMCSSFEFLAAHLWKARTRALGLRRDAMVCLQFAVDIRKRTETPLPEGFSGNAYVLASVASTARELLEELTLESIVNKIREAKKSIDQGYINSYMEALGGSNDGNLPPLKELTLISDWTKMPFHNVGFGNGGEPADYMAPLCPPVPQVAYFMKNPKDAKGVLVRIGLDPRDVNGFSNHFLDC

SEQ ID NO: 3 - DNA, 1560 bp, arabidopsis
  CDS (1)..(1560)
atgcgaaggttaggtcaccaccgacttcatgggaagacgggaggagttggaacaaaggggatggtggcgaaactgtctatcggagtcattgttctactgatctgtacgctctcgctgcttttctcggctaacatcggtagcaatcgcgaaccgacccgtccatctaagataaacgtggaagagctctgggagagtgctaaatctggtggttggagaccgtcttctgcaccacgatctgactggcctcctcctacgaaggagacaaatggttatctccgtgttcgatgtaatggtggtttgaatcagcaacgtagtgcgatatgtaatgcagtacttgctgcgcggatcatgaatgctacacttgtgttgcccgaattagatgcaaactcgttttggcatgatgacagtggttttcaaggtatctatgacgttgaacattttattgagacactgaagtatgatgttaagattgtgggaaagatccccgatgttcacaaaaatgggaaaaccaagaagataaaagcttttcagattcgtcctcctcgagatgctcctattgagtggtacctgacaactgctctaaaggcaatgagggaacatagtgctatctatcttacccctttttcacatcgtttagcggaggaaattgacaatcctgaatatcaaaggttaaggtgcagagtgaactaccatgctttgagattcaagccacacatcatgaagttgagtgaatctattgttgataaactccggtcacagggccacttcatgtcgatccatcttcgttttgaaatggatatgttggcatttgcggggtgctttgacatatttaaccctgaggaacagaagatacttaggaagtaccgtaaagaaaattttgcagataaaaggcttatttacaacgaaagacgggctatcgggaagtgtccactgacccctgaggaggtaggtcttatattacgagcaatgagattcgacaactcaacaaggatatacctagcagccggtgaactttttggcggggaacagttcatgaaaccgtttcgaacattattcccacggcttgataaccatagctcggtagacccctctgaggagctgtcagcaacctcacagggattaataggatcagctgtggattacatggtttgtcttctctccgacatttttatgccaacatatgatggaccgagcaatttcgccaacaatctcttaggtcatcgcctttactatggtttccgtaccacaatcagaccagacagaaaagcattagctccaattttcatagctagagagaaggggaaaagggctggcttcgaagaggcagtgagacgagtaatgctgaaaacaaacttcggagggcctcacaagcgtgtatctcctgaatcgttttatacaaattcatggcctgaatgtttctgtcagatgaatccgaagaagtctagtgataaatgcccgccaaataatgttatagagattcttgacagccggttagagagtatcagagatccagattcaacttctcagacgaattccacggtcactggattagaacggtaa

SEQ ID NO: 4 - protein, 519 aa, arabidopsis
MRRLGHHRLHGKTGGVGTKGMVAKLSIGVIVLLICTLSLLFSANIGSNREPTRPSKINVEELWESAKSGGWRPSSAPRSDWPPPTKETNGYLRVRCNGGLNQQRSAICNAVLAARIMNATLVLPELDANSFWHDDSGFQGIYDVEHFIETLKYDVKIVGKIPDVHKNGKTKKIKAFQIRPPRDAPIEWYLTTALKAMREHSAIYLTPFSHRLAEEIDNPEYQRLRCRVNYHALRFKPHIMKLSESIVDKLRSQGHFMSIHLRFEMDMLAFAGCFDIFNPEEQKILRKYRKENFADKRLIYNERRAIGKCPLTPEEVGLILRAMRFDNSTRIYLAAGELFGGEQFMKPFRTLFPRLDNHSSVDPSEELSATSQGLIGSAVDYMVCLLSDIFMPTYDGPSNFANNLLGHRLYYGFRTTIRPDRKALAPIFIAREKGKRAGFEEAVRRVMLKTNFGGPHKRVSPESFYTNSWPECFCQMNPKKSSDKCPPNNVIEILDSRLESIRDPDSTSQTNSTVTGLER

SEQ ID NO: 5 - DNA, 828 bp, arabidopsis
  CDS (1)..(828)
atgagtaatcccgagaagtctaaaattgctgaggaagagaatgagaattttgacgagagtgattatctttggagcgatgaagaaggagacatacgtgaaattgttcttggccttcctgcactaagacttagtgagagtctcaatgtggatgaaagcatccgtaacgaagccgtttccgcggcgaagttgatcgtggcagccgccaaggaagccacagggaatacagaacaaagtgctagtggtaccggtgacgttagtggaactccagaaatcataaaagttgggaaaccccgtgtattattggaagcgctggccgaagcatctggttgtacccctattggggtgaccactactgaaggagaggtagtcaagaaaccccgtaagaaaggttctactcagttgacacatctgccgcaaggccctcctatgtgtaatgtttgtgggaagggctttgcttcttggaaggctgtttttggacatctaaggcagcataaggatcgagggtatctaggttttcttcctccgccaacattcaatgctgctgaggaaggatttggtggagttgctactgcttcaaccggtggtgcagtagccactgctatttccgttggtgaagtccctactgcttcttccgctggtggactcgctacggcttcttccgatggtgaagggcttgtttcaagtatcggaagggcgaggattgacttaaatgccgatccagtcgaggaggaagacaaggtaattggagttggatctaagcgaaaatttgatctgaataggtcccccccaccagacggtgatgaagagaacgatgataaagtcgagtga

SEQ ID NO: 6 - protein, 275 aa, arabidopsis
MSNPEKSKIAEEENENFDESDYLWSDEEGDIREIVLGLPALRLSESLNVDESIRNEAVSAAKLIVAAAKEATGNTEQSASGTGDVSGTPEIIKVGKPRVLLEALAEASGCTPIGVTTTEGEVVKKPRKKGSTQLTHLPQGPPMCNVCGKGFASWKAVFGHLRQHKDRGYLGFLPPPTFNAAEEGFGGVATASTGGAVATAISVGEVPTASSAGGLATASSDGEGLVSSIGRARIDLNADPVEEEDKVIGVGSKRKFDLNRSPPPDGDEENDDKVE

SEQ ID NO: 7 - DNA, 927 bp, arabidopsis
  CDS (1)..(927)
atgactaatgctcaagagttggggcaagagggttttatgtggggcatatccaattctgatgattccggaggtggttgcaaaagaatcgagaaagaaccgcttccatcacatccgtctcatccgtcgccagagatccagactacgacggttaaaaagggaaaaaagagaacgaaaagaaatgataaaaaccatgaagaagaatcacctgatcatgaaatacatatatggaccgaaagagaaaggagaaagaagatgagggacatgttttctaaattacatgctttgcttccccaacttcctcctaaggcagacaaatcaacaattgtagatgaagcagtgagttcaatcaaatcccttgaacaaactttgcaaaaacttgaaatgcaaaagcttgagaagcttcagtattcttctgcctcaacaaacacaactcctactacaacttttgcttatgctccatcatcatcctcttctccaacagctctcctcacaccaatatcaaaccatcccatagacgccaccgcaacagattcttaccctcgcgcagctttcttggccgatcaggtatcttcttccagtgccgctgctgcgaacctgccttacccttgtaatgatccgatcgtgaatttcgatacttggtcctcacgtaacgtggtgctgactatctgtgggaatgaagctttcttcaatttgtgtgtccctaaacacaaaccaggggttttcacttctgtttgttacttgtttgagaagtacaacatggaggttttgtttgctaatgtctcctctaatgttttctggagcacctacgtgatccaagcacaggtaaatccgagttgtgagaatcagttactgggaaatggtcttggagttgtagacgttttcaagcaagtttctcaagaactggtgttatatttttcatctctataa

SEQ ID NO: 8 - protein, 308 aa, arabidopsis
MTNAQELGQEGFMWGISNSDDSGGGCKRIEKEPLPSHPSHPSPEIQTTTVKKGKKRTKRNDKNHEEESPDHEIHIWTERERRKKMRDMFSKLHALLPQLPPKADKSTIVDEAVSSIKSLEQTLQKLEMQKLEKLQYSSASTNTTPTTTFAYAPSSSSSPTALLTPISNHPIDATATDSYPRAAFLADQVSSSSAAAANLPYPCNDPIVNFDTWSSRNVVLTICGNEAFFNLCVPKHKPGVFTSVCYLFEKYNMEVLFANVSSNVFWSTYVIQAQVNPSCENQLLGNGLGVVDVFKQVSQELVLYFSSL

SEQ ID NO: 9 - DNA, 53 bp, Artificial Sequence
  Description of Artificial Sequence synthetic DNA
ggggacaagtttgtacaaaaaagcaggctcgatgcccatgttaatggcgacac

SEQ ID NO: 10 - DNA, 59 bp, Artificial Sequence
  Description of Artificial Sequence synthetic DNA
ggggaccactttgtacaagaaagctgggtttagcaatcaaggaaatgatttgaaaagcc

SEQ ID NO: 11 - DNA, 53 bp, Artificial Sequence
  Description of Artificial Sequence synthetic DNA
ggggacaagtttgtacaaaaaagcaggctcgatggtggcgaaactgtctatcg

SEQ ID NO: 12 - DNA, 52 bp, Artificial Sequence
  Description of Artificial Sequence synthetic DNA
ggggaccactttgtacaagaaagctgggtttaccgttctaatccagtgaccg

SEQ ID NO: 13 - DNA, 59 bp, Artificial Sequence
  Description of Artificial Sequence synthetic DNA
ggggacaagtttgtacaaaaaagcaggctcgatgagtaatcccgagaagtctaaaattg

SEQ ID NO: 14 - DNA, 55 bp, Artificial Sequence
  Description of Artificial Sequence synthetic DNA
ggggaccactttgtacaagaaagctgggttcactcgactttatcatcgttctctt

SEQ ID NO: 15 - DNA, 53 bp, Artificial Sequence
  Description of Artificial Sequence synthetic DNA
ggggacaagtttgtacaaaaaagcaggctatgactaatgctcaagagttgggg

SEQ ID NO: 16 - DNA, 54 bp, Artificial Sequence
  Description of Artificial Sequence synthetic DNA
ggggaccactttgtacaagaaagctgggtttaaagtgaaaagtccaagctaatc

SEQ ID NO: 17 - DNA, 25 bp, Artificial Sequence
  LB2 Primer
tgaccatcatacccattgctgatcc